(19) United States
(12) Patent Application Publication
(10) Pub. No.: US 2015/0272465 A1
(43) Pub. Date: Oct. 1, 2015

(54) SYSTEMS AND METHODS FOR PORTABLE NEUROFEEDBACK

(71) Applicant: Sharp Laboratories of America, Inc., Camas, WA (US)

(72) Inventor: Atsushi Ishii, Vancouver, WA (US)

(73) Assignee: Sharp Laboratories of America, Inc., Camas, WA (US)

(21) Appl. No.: 14/231,399

(22) Filed: Mar. 31, 2014

(51) Int. Cl. A61B 5/0482; A61B 5/00; A61B 5/0478

(52) U.S. Cl. CPC A61B 5/0482; A61B 5/0478; A61B 5/486

(57) ABSTRACT

A portable neurofeedback device is described. The neurofeedback device includes a wearable fixture. One or more brainwave sensors are attached to the wearable fixture. A processing unit is attached to the wearable fixture. The processing unit receives brainwave signals captured by the one or more brainwave sensors. The processing unit computes one or more user-feedback values that represent current brain activity. A display module is attached to the wearable fixture that displays a visual representation of the one or more user-feedback values.

### TECHNICAL FIELD

The present disclosure relates generally to neurofeedback. More specifically, the present disclosure relates to systems and methods for portable neurofeedback.

### BACKGROUND

The use of electronic devices has become commonplace in modern society. Electronic devices have become smaller and more powerful in order to meet consumer needs and to improve portability and convenience. Electronic devices that are commonly used include computing devices, such as personal computers or laptops. Other electronic devices commonly used include cellular phones, smart phones, Personal Digital Assistants, tablet devices, netbooks, e-readers, Global Positioning Satellite (GPS) units, etc. Consumers have become dependent upon electronic devices and have come to expect increased functionality.

Neurofeedback is a type of biofeedback that uses electronic instruments to capture and feedback brain activity to a user for teaching self-regulation. Typically, neurofeedback is performed in a clinical or laboratory setting where a user is confined to the location of the neurofeedback instruments. However, a portable neurofeedback device may allow a user to perform neurofeedback outside of clinics or laboratories. As can be seen from this discussion, systems and methods that improve the portability and privacy of neurofeedback may be beneficial.

### BRIEF DESCRIPTION OF THE DRAWINGS

FIG. 1 is a block diagram illustrating one configuration of a neurofeedback device;

FIG. 2 is a flow diagram illustrating one implementation of a method for performing portable neurofeedback;

FIG. 3 illustrates one configuration of a neurofeedback system;

FIG. 4 illustrates one implementation of a portable neurofeedback device;

FIG. 5 is a block diagram illustrating a detailed configuration of a portable neurofeedback device;

FIG. 6 is a block diagram illustrating one configuration of a signal processor in accordance with the described systems and methods;

FIG. 7 illustrates another implementation of a portable neurofeedback device;

FIG. 8 illustrates various components that may be utilized in a neurofeedback device; and

FIG. 9 is a block diagram illustrating another configuration of a neurofeedback device in which systems and methods for portable neurofeedback may be implemented

### DETAILED DESCRIPTION

A portable neurofeedback device is described. The neurofeedback device includes a wearable fixture. One or more brainwave sensors are attached to the wearable fixture. A processing unit is attached to the wearable fixture. The processing unit receives brainwave signals captured by the one or more brainwave sensors. The processing unit computes one or more user-feedback values that represent current brain activity. A display module is attached to the wearable fixture that displays a visual representation of the one or more user-feedback values.

The visual representation of the one or more user-feedback values may be displayed in real time. The user-feedback value may correspond to a power ratio of two or more frequency bands.

The display module may only be visible to a user wearing the neurofeedback device. The display module may include at least one of a light emitting diode (LED), an LED array, a multi-color LED array and a liquid crystal display (LCD).

Multiple algorithms may be used by the processing unit to compute the one or more user-feedback values. Each algorithm may produce a different user-feedback value. The multiple algorithms may correspond to different types of neurofeedback training. A selection means may be attached to the neurofeedback device for selecting one or more of the multiple algorithms.

The neurofeedback device may also include one or more communication interfaces to communicate with at least one external device. The at least one external device may display a visual representation of the one or more user-feedback values and may save log data. The at least one external device may further include a user-interface for configuring the neurofeedback device.

The wearable fixture may be a cap that includes a crown portion. The crown portion may include the one or more brainwave sensors. The wearable fixture may also include a brim portion that projects from the crown portion. The display module may be attached to a bottom side of the brim portion and may be visible by a user wearing the neurofeedback device.

The wearable fixture may include a headband portion. The headband portion may include the one or more brainwave sensors. The wearable fixture may also include an eyeglass portion. The display module may be attached to the eyeglass portion and may be visible by a user wearing the neurofeedback device.

A method for performing neurofeedback by a portable neurofeedback device is also described. The method includes capturing, by one or more brainwave sensors attached to a wearable fixture, brainwave signals. The method also includes computing, by a processing unit attached to the wearable fixture, one or more user-feedback values that represent current brain activity. The method further includes displaying, by a display module attached to the wearable fixture, a visual representation of the one or more user-feedback values.

Various examples of the systems and methods disclosed herein are now described with reference to the figures, where like reference numbers may indicate functionally similar elements. The systems and methods as generally described and illustrated in the figures herein could be arranged and designed in a wide variety of different implementations. Thus, the following more detailed description of several implementations, as represented in the figures, is not intended to limit scope, as claimed, but is merely representative of the systems and methods.

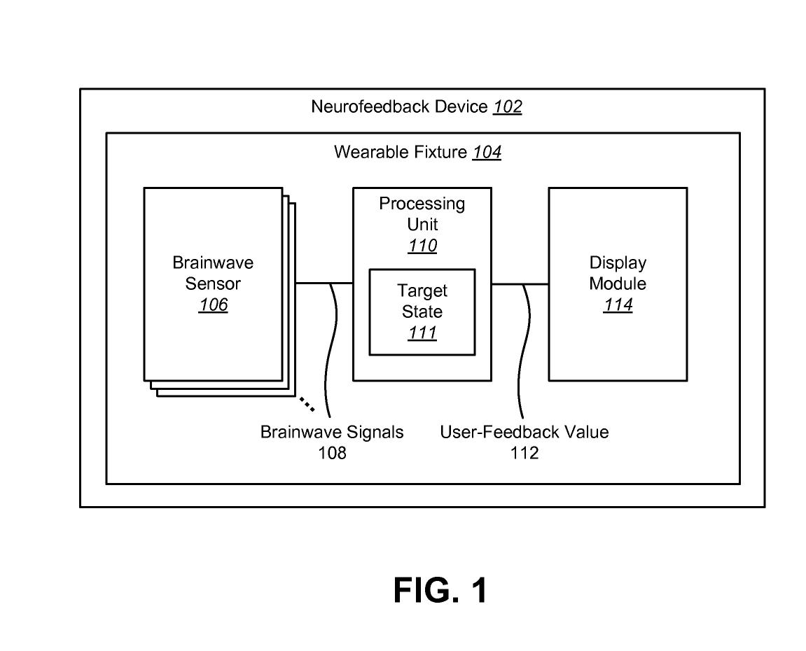

FIG. 1 is a block diagram illustrating one configuration of a neurofeedback device 102. Neurofeedback is a type of biofeedback that uses instruments to capture and feedback brain activity to a user for teaching self-regulation. In some configurations, neurofeedback may capture brainwaves using electroencephalography (EEG), magnetoencephalography (MEG) or other neuroimaging techniques. Neurofeedback may provide a real-time display of a signal corresponding to the captured brainwaves. Neurofeedback is used in medical fields that may include the clinical treatment of attention deficit hyperactivity disorder (ADHD), addiction, anxiety, autism and depression. Neurofeedback may also be used in non-medical applications that include artistic and athletic performance enhancements.

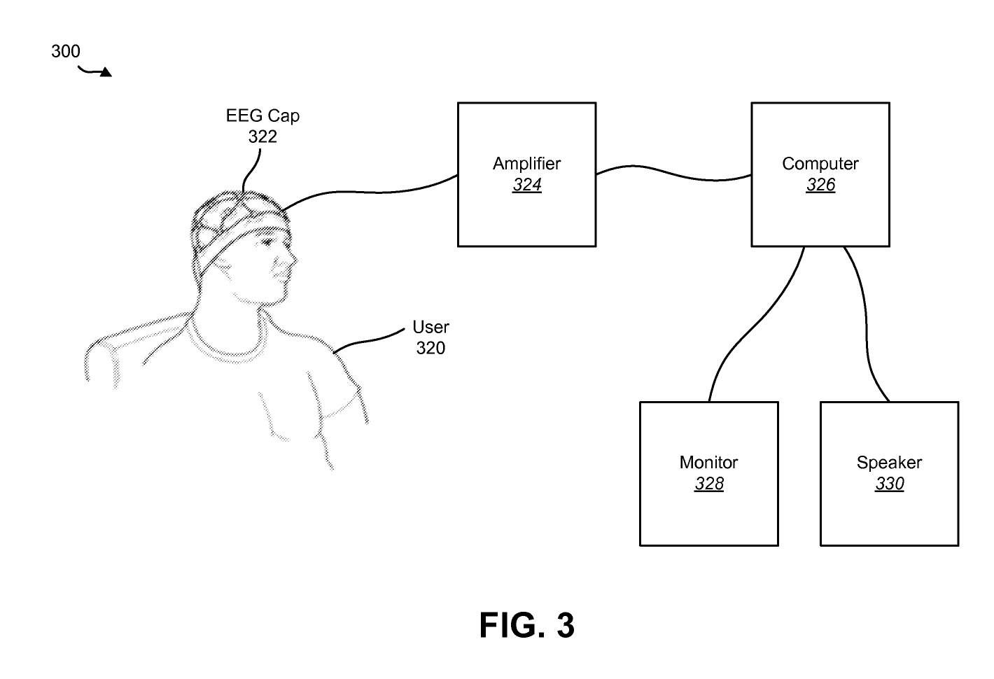

In one approach to neurofeedback, EEG electrodes are applied to the scalp of a user to capture brainwave signals. The EEG electrodes may be fitted in a cap. An amplifier may convert the analog signals to digitized data streams. A computer receives the digital signals and performs signal processing to generate real-time information. The computer displays visual feedback via a monitor screen and provides audible feedback via a speaker. An example of this approach to neurofeedback is described below in connection with FIG. 3.

This approach to neurofeedback is used in a clinical or laboratory environment. Therapists or researchers provide neurofeedback training sessions to subjects. However, this approach is neither portable nor wearable. Portability and wearability are required to perform neurofeedback outside of clinics or laboratories. For example, an artistic performer or an athlete may benefit from neurofeedback-based self-regulation during a performance.

The systems and methods described herein provide a portable and wearable approach to neurofeedback. A neurofeedback device 102 may include one or more wearable fixtures 104. The wearable fixture 104 may be a structure that is worn by or attached to a user of the neurofeedback device 102. The form factor of the neurofeedback device 102 may be anything that holds at least one or more brainwave sensors 106, a processing unit 110 and a display module 114 in a portable and wearable manner.

The neurofeedback device 102 may be a single unit (e.g., piece) or multiple units. In one configuration, the wearable fixture 104 may be a single unit that is worn on or attached to the head of a user. For example, the wearable fixture 104 may be a head covering (e.g., a cap, hat, helmet, etc.), headgear, headpiece or headband. In another configuration, the wearable fixture 104 may include two or more separate units that may include different components of the neurofeedback device 102. One example is a headband with EEG sensors and eye glasses with a display (e.g., light emitting diodes (LEDs)). Another example is an EEG cap and a bracelet with a liquid crystal display (LCD).

The brainwave sensors 106 may be attached to the wearable fixture 104. In one configuration, the brainwave sensors 106 may include non-invasive EEG electrodes. The brainwave sensors 106 may contact the scalp of the user wearing the neurofeedback device 102. The brainwave sensors 106 may detect oscillations of brain electric potentials. Each brainwave sensor 106 may capture a brainwave signal 108. In one configuration, the brainwave signal 108 may be a voltage (e.g., electric potential difference). The voltage may be the difference between the electric potential at a brainwave sensor 106 and a reference point. In another configuration, the brainwave sensors 106 may include functional near infrared spectroscopy (fNIRS) sensors.

The processing unit 110 may be attached to the wearable fixture 104. The processing unit 110 may be a computer system that includes a processor, memory and instructions stored in the memory that may be executed by the processor. The processing unit 110 may be an embedded system.

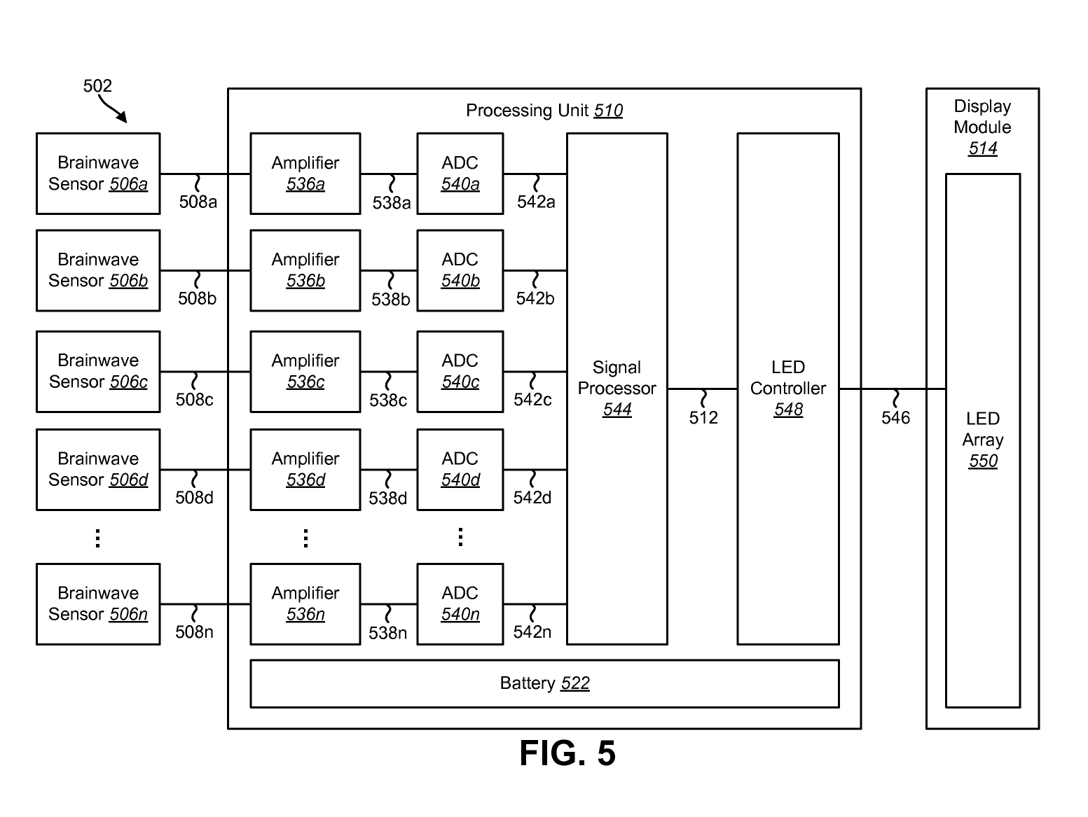

The processing unit 110 may be coupled to the one or more brainwave sensors 106. The processing unit 110 may receive the brainwave signals 108 captured by the one or more brainwave sensors 106. The processing unit 110 may compute one or more user-feedback values 112 based on the brainwave signals 108. The one or more user-feedback values 112 may represent current brain activity. One configuration of the processing unit 110 is described in connection with FIG. 5.

The neurofeedback device 102 may also include a display module 114. The display module 114 may be attached to the wearable fixture 104. The display module 114 may display a visual representation of the one or more user-feedback values 112. The display module 114 may include one or more means for displaying visual information. For example, the display module 114 may include a light emitting diode (LED), an LED array, a multi-color LED array, a liquid crystal display (LCD) or other structure that conveys visual information. The display module 114 may be wired or wirelessly coupled to the processing unit 110.

In one configuration, the display module 114 is only visible to a user wearing the neurofeedback device 102. In this configuration, the display module 114 may be housed in a discrete location on the wearable fixture 104 that is visible to the user of the neurofeedback device 102 but is not easily viewed by others. Therefore, the visual representation of the one or more user-feedback values 112 may be concealed from being observed by all except for the user. For example, the display module 114 may be located on the underside of the brim of a cap, or on the inside of glasses worn by the user. This may ensure the privacy of the user.

In another configuration, the display module 114 may be visible to others, not just the user wearing the neurofeedback device 102. In this configuration, the visual representation of the one or more user-feedback values 112 is not concealed from view. For example, the display module 114 may be located on a bracelet or armband worn by the user.

Brain activity may exhibit neural oscillation, which may be characterized by frequency, amplitude and phase. In one configuration, the frequency of the neural oscillation may be measured using EEG. The neural oscillation may be associated with specific brainwave frequency bands. These brainwave frequency bands include alpha (8-13 Hertz (Hz)), beta (13-30 Hz), gamma (25-100 Hz), theta (4-8 Hz) and delta (0.1-4 Hz). The brainwave frequency bands are associated with different brain states. For example, delta activity is dominant during deep sleep. Alpha, mixed with low amplitude delta, theta and beta, is typically predominant in awake-resting states. Different combinations of these brainwave frequency bands may be associated with different behavioral or cognitive states.

For self-regulation training, a user may augment certain brainwave frequency bands while inhibiting other brainwave frequency bands to reach a target state 111. For example, users may be able to augment strengths of certain brainwave frequency bands by training. By presenting accurate measurements of physiological activities as feedback, over time a user may learn how to change thinking, emotions, and/or behavior to set the physiological state in a desired target state 111. The target state 111 may correspond to certain combinations of brainwave frequency bands. Whether the user reaches the target state 111 may be determined by analyzing measured power levels of the brainwave signals 108. The user may perform different types of neurofeedback training to reach different target states 111.

Alternatively, for self-regulation training, the neurofeedback device 102 may run one or more training programs (e.g., games). The user may be rewarded when successfully achieving certain target states 111. The rewards may be visually indicated to the user by the display module 114.

Multiple algorithms may be used by the processing unit 110 to compute the one or more user-feedback values 112. Each algorithm may correspond to a different target state 111 and may produce a different user-feedback value 112. Therefore, the multiple algorithms correspond to different types of neurofeedback training. For instance, for neurofeedback training to enhance attention, the processing unit 110 may generate at least one user-feedback value 112 reflecting beta wave strength relative to the other lower frequency bands. Alternatively, instead of low-level band powers, the processing unit 110 may generate at least one user-feedback value 112 corresponding to high-level mental states (e.g., relaxation, focus, cognitive load, emotions, etc.). In one configuration, the user-feedback value 112 may correspond to a power ratio of two or more frequency bands.

The type of neurofeedback training that is performed by the neurofeedback device 102 may be selectable. In one configuration, the neurofeedback device 102 may include one or more selection means for selecting one or more of the multiple algorithms. The selection means may be a user interface that is attached to the neurofeedback device 102. For example, the selection means may be a dial, button, switch, touch screen or other user interface through which a user may select an algorithm corresponding to a desired neurofeedback training type.

In one configuration, the neurofeedback device 102 may be configured to provide visual feedback for a target state 111. The neurofeedback device 102 may provide a visual indication of whether the user successfully achieves or fails to achieve the target state 111. The user-feedback value 112 computed by the processing unit 110 may indicate the success or failure (or degree of success or failure) to achieve a target state 111.

The display module 114 may provide visual feedback of the current brain activity in real time. The visual feedback may correspond to the user-feedback value 112 computed by the processing unit 110. In one example, the display module 114 may include a single LED that lights up when a target state 111 is achieved and turns off when the target state 111 is not achieved, or vice versa. In another example, the display module 114 may include an array of LEDs that may light up in sequence to indicate an amount (e.g., degree) of success or failure to achieve a target state 111. In yet another example, the display module 114 may include a screen (e.g., an LCD screen) that may display bar graphs presenting powers of brainwave frequency bands or other visual feedback of current brain activity.

The neurofeedback device 102 may also include a communications interface to communicate with at least one external device. The communication interface may provide for wired or wireless communication with one or more external devices (e.g., personal computers, smartphones, tablets, etc.). The communication interface may allow the external device to display computed results and save log data. For example, the external device may display the one or more user-feedback values 112 or a visual representation based on the user-feedback values 112.

An external device may also communicate with the neurofeedback device 102 (via the communication interface) to configure the neurofeedback device 102. For example, the external device may have a user-interface that may be used to configure the neurofeedback device 102. In one configuration, the algorithm used by the processing unit 110 to compute the one or more user-feedback values 112 may be selected through the user-interface on the external device.

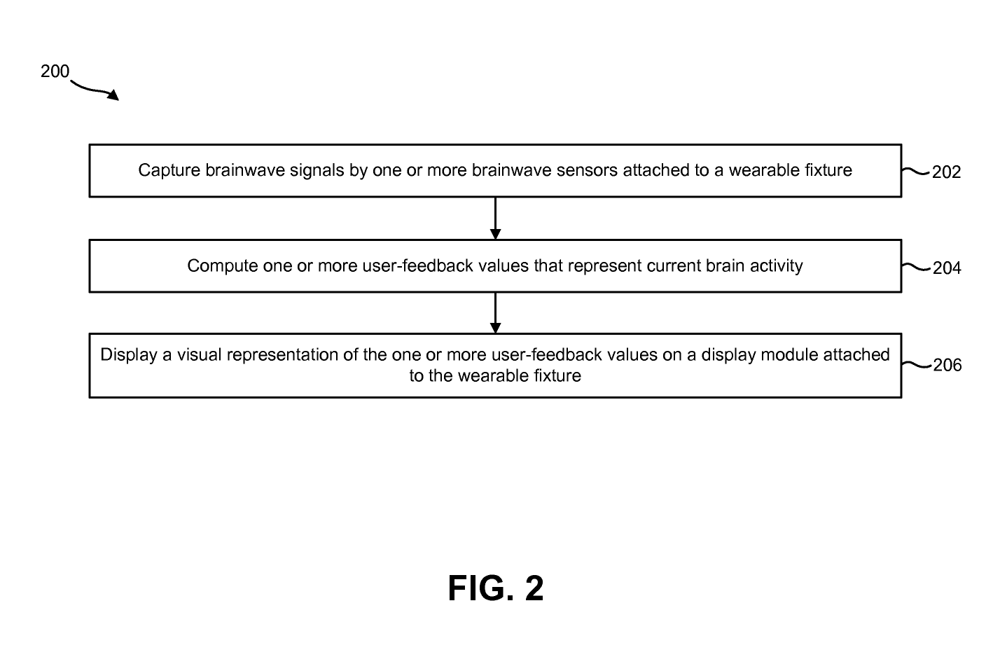

FIG. 2 is a flow diagram illustrating one implementation of a method 200 for performing portable neurofeedback. The method 200 may be performed by a neurofeedback device 102 described above in connection with FIG. 1.

The neurofeedback device 102 may capture 202 brainwave signals 108. In one configuration, the neurofeedback device 102 may have one or more brainwave sensors 106 attached to a wearable fixture 104. The wearable fixture 104 may be a structure that is worn by or attached to a user of the neurofeedback device 102. The wearable fixture 104 may be a single unit that attaches to the body of a user. Alternatively, the wearable fixture 104 may be multiple units that attach in multiple locations on the body of a user.

In one configuration, the brainwave sensors 106 may include EEG electrodes. The brainwave sensors 106 may contact the scalp of the user wearing the neurofeedback device 102. The brainwave sensors 106 may detect oscillations of brain electric potentials. Each brainwave sensor 106 may capture a brainwave signal 108. In one configuration, the brainwave signal 108 may be a voltage (e.g., electric potential difference).

The neurofeedback device 102 may compute 204 one or more user feedback values 112 that represent current brain activity. For example, a processing unit 110 attached to the wearable fixture 104 may receive the brainwave signals 108 captured by the one or more brainwave sensors 106. Multiple algorithms may be used by the processing unit 110 to compute the one or more user-feedback values 112. Each algorithm may correspond to a different target state 111 and may produce a different user-feedback value 112. The target state 111 may correspond to certain combinations of brainwave frequency bands (e.g., alpha, beta, gamma, theta, delta, etc.). The neurofeedback device 102 may determine whether the user reaches a target state 111 based on measured power levels of the brainwave signals 108.

In one configuration, the computed user-feedback value 112 may correspond to a power ratio of two or more frequency bands. For example, the user-feedback value 112 may be the ratio of the theta frequency band to the alpha frequency band. In another example, the user-feedback value 112 may correspond to a power ratio of the beta frequency band to other lower frequency bands.

The neurofeedback device 102 may display 206 a visual representation of the one or more user-feedback values 112. The visual representation of the user-feedback values 112 may be displayed 206 on a display module 114 attached to the wearable fixture 104. The display module 114 may include one or more means for displaying visual information. For example, the display module 114 may include a light emitting diode (LED), an LED array, a multi-color LED array, a liquid crystal display (LCD) or other structure that conveys visual information.

The visual indication displayed 206 by the neurofeedback device 102 may indicate whether the user successfully achieves or fails to achieve the target state 111. The user-feedback value 112 computed by the neurofeedback device 102 may indicate the success or failure (or degree of success or failure) to achieve the target state 111 in real time. Therefore, the neurofeedback device 102 may provide visual feedback of current brain activity in real time.

The visual feedback may correspond to the user-feedback value 112 computed by the processing unit 110. In one example, the display module 114 may include a single LED that lights up when a target state 111 is achieved and turns off when the target state 111 is not achieved, or vice versa. In another example, the display module 114 may include an array of LEDs that may light up in sequence to indicate an amount (e.g., degree) of success or failure to achieve a target state 111. In yet another example, the display module 114 may include a screen (e.g., an LCD screen) that may display bar graphs presenting powers of brainwave frequency bands or other visual feedback of current brain activity.

In one configuration, the neurofeedback device 102 may display 206 the visual representation of the one or more user-feedback values 112 only to the user wearing the neurofeedback device 102. For example, the display module 114 may be housed in a structure on the wearable fixture 104 that is visible to the user but shields the display module 114 from others. In another configuration, the display module 114 may be visible to others, not just the user wearing the neurofeedback device 102.

FIG. 3 illustrates one configuration of a neurofeedback system 300. The neurofeedback system 300 illustrated in FIG. 3 is a known approach to neurofeedback in a clinical or laboratory setting. The neurofeedback system 300 uses EEG electrodes to measure brain activity. A user 320 wears an EEG cap 322 that includes EEG electrodes. An EEG amplifier 324 collects analog signals from the EEG electrodes. The analog signals may be voltages associated with the EEG electrodes. The EEG amplifier 324 converts the analog signals to digitized data streams, performs signal processing and generates real-time information, such as time-domain waveforms and power spectral densities.

A computer 326 may receive the real-time information from the amplifier 324. Based on the real-time information, the computer 326 runs application programs to present visual feedback in the monitor screen 328. Optionally, the computer 326 may provide audible feedback via one or more speakers 330.

The visual feedback on the monitor screen 328 may be realized by bar graphs presenting powers of brainwave frequency bands (e.g., alpha, beta, gamma, theta, delta, etc.). For self-regulation training, the user 320 is instructed to attempt augmenting certain brainwave frequency bands and/or inhibiting other brainwave frequency bands to reach a certain target state 111. As discussed above, a target state 111 may be a combination or ratio of brainwave frequency band strengths. When the user 320 successfully achieves or fails to achieve the target state 111, the computer 326 produces visual/audible indications.

Alternatively, the computer 326 may run entertainment programs (e.g., games) where the user 320 obtains rewards (e.g., points, etc.) when successfully achieving certain criteria, such as the target state 111. The rewards are visually/audibly indicated to the user 320 by the monitor screen 328 and/or speaker 330.

This type of neurofeedback system 300 is typically used in the clinical or laboratory environment, where therapists or researchers provide neurofeedback training sessions to subjects. However, this neurofeedback system 300 is neither portable nor wearable. Portability and wearability are required for use of neurofeedback outside of clinics or laboratories. For example, when an artistic performer or an athlete desires to do self-regulation during a performance, a portable neurofeedback device 102 may be beneficial. Therefore, benefits may be realized by the systems and methods for portable neurofeedback described herein.

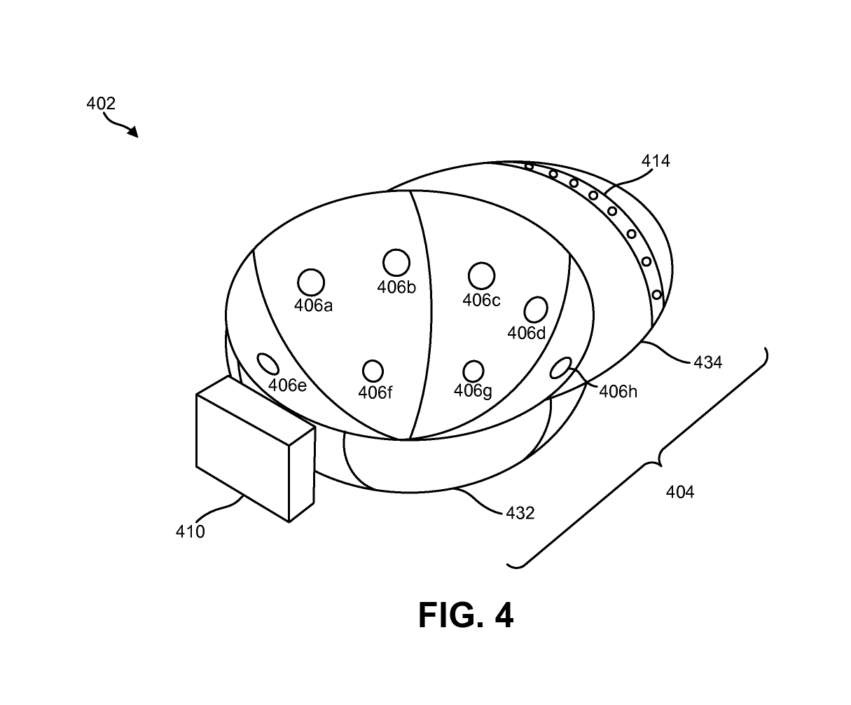

FIG. 4 illustrates one implementation of a portable neurofeedback device 402. In this implementation, the wearable fixture 404 is a cap. The neurofeedback device 402 is a more detailed configuration of the neurofeedback device 102 described above in connection with FIG. 1.

The neurofeedback device 402 may be worn by a user and is portable. For example, the neurofeedback device 402 may be worn on the head of a user to provide neurofeedback outside a clinical or laboratory setting. FIG. 4 illustrates the underside of the neurofeedback device 402. In other words, the inside cavity of the cap is visible in FIG. 4.

The wearable fixture 404 may include a crown portion 432. The wearable fixture 404 may also include a brim portion 434 that projects from the crown portion 432. The crown portion 432 may include one or more brainwave sensors 406. It should be noted that a plurality of brainwave sensors 406a-h are depicted in FIG. 4. However, any number of brainwave sensors 406 may be used. In one configuration, the brainwave sensors 406a-h may be EEG electrodes. The brainwave sensors 406a-h may be attached to the inside of the crown portion 432 of the cap such that the brainwave sensors 406a-h may contact the scalp of the user wearing the neurofeedback device 402. The brainwave sensors 406a-h may detect oscillations of brain electric potentials. Each brainwave sensor 406 may capture a brainwave signal 108.

The neurofeedback device 402 may include a processing unit 410. In one configuration, the processing unit 410 may be attached to the crown portion 432 of the cap. It should be noted that the processing unit 410 may be attached to another portion of the neurofeedback device 402 (e.g., the brim portion 434). The processing unit 410 may be coupled (e.g., via wire or wirelessly) to the brainwave sensors 406a-h to receive the brainwave signal 108. The processing unit 410 may compute one or more user-feedback values 112 that represent current brain activity based on the received brainwave signals 108. This may be accomplished as described in connection with FIG. 5.

The neurofeedback device 402 also includes a display module 414. The display module 414 may be attached to the bottom side of the brim portion 434. The display module 414 is coupled to (e.g., via wire or wirelessly) the processing unit 410. The display module 414 may be an LED array that includes a plurality of LEDs. The display module 414 may display a visual representation of the one or more user-feedback values. This may be accomplished as described in connection with FIG. 5.

The display module 414 may be visible to a user wearing the neurofeedback device 402. In one configuration, the display module 414 is oriented such that the light from the LED array may only be seen by the user wearing the neurofeedback device 402. In another configuration, the display module 414 may additionally be housed in a structure that blocks or obscures the visibility of the display module 414 to all except the user wearing the neurofeedback device 402. This may prevent others from viewing the user's mental states and may ensure privacy.

The neurofeedback device 402 may further include one or more batteries to provide power to the electrical components. The one or more batteries may be housed in the processing unit 410 or may be located elsewhere on the neurofeedback device 402.

FIG. 5 is a block diagram illustrating a detailed configuration of a portable neurofeedback device 502. The neurofeedback device 502 is a more detailed configuration of the neurofeedback device 102 described above in connection with FIG. 1. The neurofeedback device 502 may include multiple brainwave sensors 506a-n, a processing unit 510 and a display module 514. The neurofeedback device 502 may include a wearable fixture 104 (not shown for clarity) on which the one or more brainwave sensors 506, processing unit 510 and display module 514 are attached.

The processing unit 510 may include one or more amplifiers 536, one or more analog-to-digital converters (ADCs) 540, a signal processor 544, an LED controller 548 and one or more batteries 552. A pair of an amplifier 536 and an ADC 540 may correspond to each brainwave sensor 506 that is coupled to the processing unit 510. As illustrated in FIG. 5, the neurofeedback device 502 may include a plurality of brainwave sensors 506a-n. Therefore, the processing unit 510 may include a corresponding number of amplifiers 536a-n and ADCs 540a-n. It should be noted that while multiple brainwave sensors 506a-n, amplifiers 536a-n and ADCs 540a-n are depicted in FIG. 5, the neurofeedback device 502 may include at least one brainwave sensor 506 and a corresponding amplifier 536 and ADC 540.

The amplifiers 536a-n may receive brainwave signals 508a-n that are captured by the brainwave sensors 506a-n coupled to the amplifiers 536a-n. In one configuration, the brainwave sensors 506a-n may be EEG sensors that produce an analog voltage signal (e.g., the brainwave signal 508). The amplifiers 536a-n may amplify the brainwave signals 508a-n from the brainwave sensors 506a-n. The amplifiers 536a-n may also perform other analog signal processing (e.g., filtering) to produce amplified signals 538a-n. The ADCs 540a-n may convert the analog input (e.g., the amplified signals 538a-n) from the amplifiers 536a-n to digitized brainwave signals 542a-n by sampling and quantization.

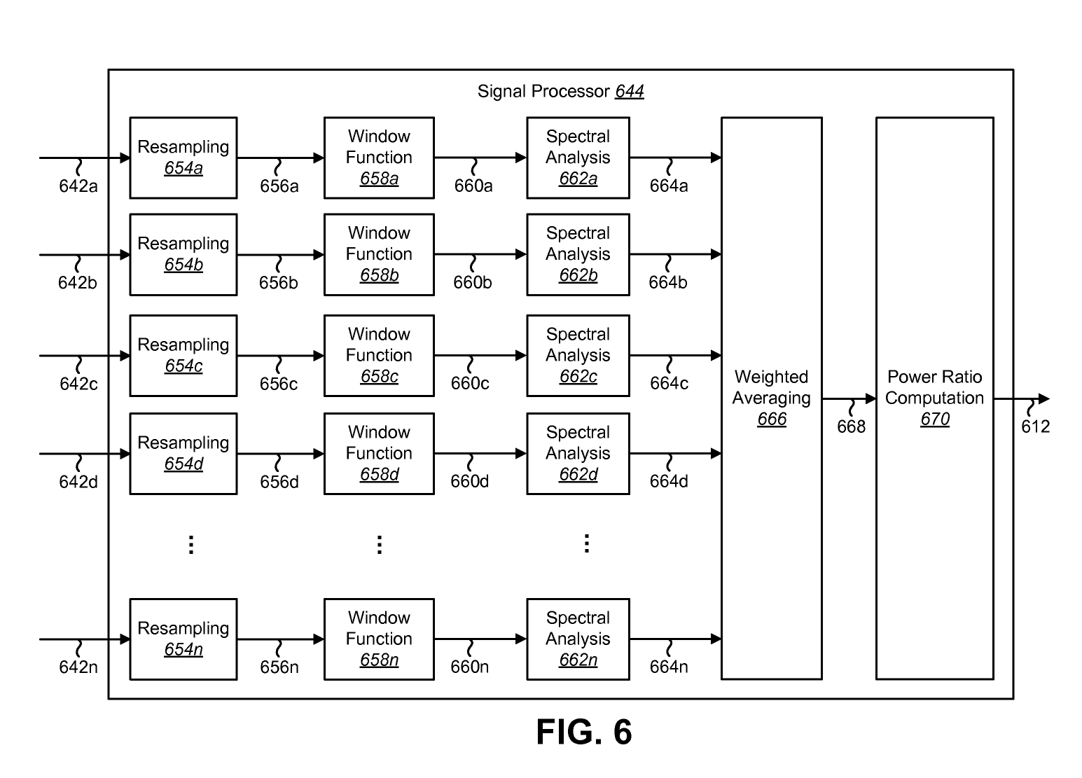

The signal processor 544 may collect the digitized brainwave signals 542a-n from the ADCs 540a-n. The signal processor 544 may perform digital signal processing to analyze the digitized brainwave signals 542a-n. This may be accomplished as described in connection with FIG. 6. Based on the signal processing, the signal processor 544 may generate at least one user-feedback value 512 that indicates the brainwave or mental state in a real-time manner. The user-feedback value 512 may be an index or indices. The form of the index/indices may depend on the type of neurofeedback training that is configured.

The processing unit 510 may include one or more algorithms that are used by the signal processor 544 to determine the user-feedback value 512. The neurofeedback device 502 may include a selection means for selecting one or more of the multiple algorithms. The selection means may be a user interface that is attached to the neurofeedback device 102. For example, the selection means may be a dial, button, switch, touch screen or other user interface through which a user may select an algorithm corresponding to a desired neurofeedback training type. Additionally, if an external device is connected to the neurofeedback device 502, the external device may have a user interface to select one or more of the multiple algorithms. In this case, some messages or commands may be sent from the external device to the processing unit 510 to configure the signal processor 544 with the selected algorithm.

The LED controller 548 may provide a control signal 546 to the display module 514 to display a visual representation based on the one or more user-feedback values 512. The display module 514 may include an LED array 550. The control signal 546 may drive the LEDs built into the LED array 550. The LED controller 548 may receive the user-feedback value 512 from the signal processor 544. The LED controller 548 may encode the control signal 546 based on the user-feedback value 512. The control signal 546 may indicate how to light the LEDs of the LED array 550. For example, the control signal 546 may indicate the sequence of which LEDs are illuminated and which LEDs are not illuminated.

The neurofeedback device 502 may further include one or more batteries 552 to provide power to the electrical components. The one or more batteries may be housed in the processing unit 510 or may be located elsewhere on the neurofeedback device 502.

FIG. 6 is a block diagram illustrating one configuration of a signal processor 644 in accordance with the described systems and methods. The signal processor 644 may be included in a processing unit 510 as described above in connection with FIG. 5. FIG. 6 further illustrates one or more algorithms for computing a user-feedback value 612.

The configuration illustrated in FIG. 6 may be used in different neurofeedback training applications. For example, an alpha-theta neurofeedback training application may be implemented. In general, the alpha-theta neurofeedback training involves procedures to instruct a user of the neurofeedback device 102 to put themselves into states of deep relaxation and to maximize the power ratio of the theta frequency band to the alpha frequency band by self-regulation.

The signal processor 644 may receive one or more digitized brainwave signals 642a-n. The digitized brainwave signals 642a-n may be produced by one or more ADCs 540, as described above in connection with FIG. 5. It should be noted that while multiple digitized brainwave signals 642a-n are depicted in FIG. 6, the neurofeedback device 102 may include at least one brainwave sensor 106 and, therefore, at least one digitized brainwave signal 642 may be received by the signal processor 644.

Resampling modules 654a-n may receive the digitized brainwave signals 642a-n. In one configuration, each digitized brainwave signal 642a-n may be re-sampled with a pre-determined sampling rate in a corresponding resampling module 654a-n.

The re-sampled signals 656a-n may be received by a corresponding window function module 658a-n. In one configuration, each window function module 658a-n may group the re-sampled signals 656a-n into data sets 660a-n with sliding time windows.

The data sets 660a-n may be received by corresponding spectral analysis modules 662a-n. In one configuration, the spectral analysis modules 662a-n may compute a power spectrum density 664a-n of each data set 660a-n using a power spectrum density algorithm such as Discrete Fourier Transform (DFT) and auto-regression.

A weighted averaging module 666 may receive the power spectrum densities 664a-n. For a given time window, multiple streams of the power spectrum densities 664a-n may be averaged by the weighted averaging module 666 using a weight function to produce an averaged power spectrum density 668. The weight function may take into account that the strength of a certain frequency band may differ depending on the locations on the scalp. In some implementations, a pre-determined weight function may be used to average the power spectrum density 664a-n derived from multiple brainwave sensors 106. In another implementation, the weight function may be adjusted during a calibration process.

A power ratio computation module 670 may receive the averaged power spectrum density 668. The power ratio computation module 670 may use the averaged power spectrum density 668 to compute a power ratio 612 of two or more frequency bands. For example, the power ratio computation module 670 may compute a power ratio of the theta frequency band to the alpha frequency band.

In one configuration, the power ratio 612 may be computed using fast Fourier transform (FFT) or other methods that produce spectrum power density. These methods may provide the energy of each frequency block (e.g., 1-2 Hz, 2-3 Hz, 3-4 Hz, etc.). One example of a power ratio 612 is a theta/alpha power ratio 612. For the alpha band power, the energies for all the frequency blocks corresponding to the alpha band are summed. For the theta band power, the energies for all the frequency blocks corresponding to the theta band are summed. The theta/alpha power ratio 612 may then be computed as the total theta band energy divided by the total alpha band energy. Other power ratios 612 may be computed similarly.

The power ratio 612 may be used as the user-feedback value 112, as discussed in connection with FIG. 1. In another implementation, the power ratio 612 may be used as the index value to be displayed on the LED Array 550, as discussed in connection with FIG. 5.

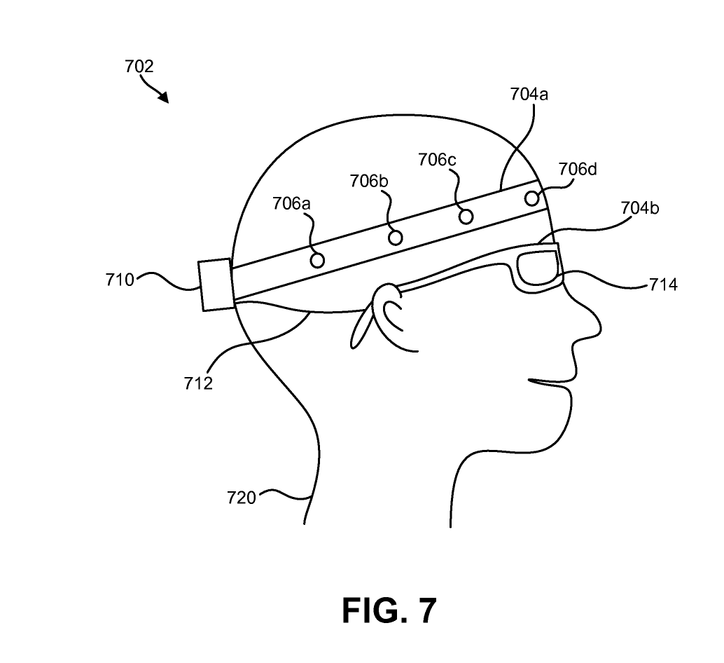

FIG. 7 illustrates another implementation of a portable neurofeedback device 702. The neurofeedback device 702 is a more detailed configuration of the neurofeedback device 102 described above in connection with FIG. 1. In this implementation, the wearable fixture 704 includes two pieces: a headband portion 704a and an eyeglass portion 704b. The neurofeedback device 702 may be worn by a user 720 and is portable. In other words, the neurofeedback device 702 may be worn by a user 720 to provide neurofeedback outside a clinical or laboratory setting.

The headband portion 704a and the eyeglass portion 704b may be worn on the head of a user 720. The headband portion 704a may include one or more brainwave sensors 706a-d. It should be noted that a plurality of brainwave sensors 706a-d are depicted in FIG. 7. However, any number of brainwave sensors 706 may be used. In one configuration, the brainwave sensors 706a-d may be EEG electrodes.

The brainwave sensors 706a-d may be attached to the inside of the headband portion 704a such that the brainwave sensors 706a-d may contact the scalp of the user 720 wearing the headband portion 704a. The brainwave sensors 706a-d may detect oscillations of brain electric potentials. Each brainwave sensor 706 may capture a brainwave signal 108. This may be accomplished as described above in connection with FIG. 1.

The neurofeedback device 702 may include a processing unit 710. In one configuration, the processing unit 710 may be attached to the headband portion 704a. It should be noted that the processing unit 710 may be attached to another portion of the neurofeedback device 702 (e.g., the eyeglass portion 704b). The processing unit 710 may be coupled (e.g., via wire or wirelessly) to the brainwave sensors 706a-d to receive the brainwave signal 108. The processing unit 710 may compute one or more user-feedback values 712 that represent current brain activity based on the received brainwave signals 108. This may be accomplished as described in connection with FIG. 5.

The neurofeedback device 702 also includes a display module 714. The display module 714 may be attached to the eyeglass portion 704b. In one configuration, the display module 714 is attached to the inside of the eyeglass portion 704b to allow the user 720 to view the display module 714. The display module 714 is coupled to (e.g., via wire or wirelessly) the processing unit 710. The display module 714 may display a visual representation of the one or more user-feedback values 712. This may be accomplished as described in connection with FIG. 5. The display module 714 may include one or more of an LED, an LED array, a multi-color LED array, an LCD display, or other structure that may convey visual information to the user 720.

The display module 714 may be visible to the user 720 wearing the neurofeedback device 702 but not visible by others. For example, if the display module 714 is located on the inside of the eyeglass portion 704b, the user 720 may view the visual feedback on the display module 714 but others may not see what is displayed. This may prevent others from viewing the user's 720 mental states and may ensure privacy.

The neurofeedback device 702 may further include one or more batteries to provide power to the electrical components. The one or more batteries may be housed in the processing unit 710 or may be located elsewhere on the neurofeedback device 702.

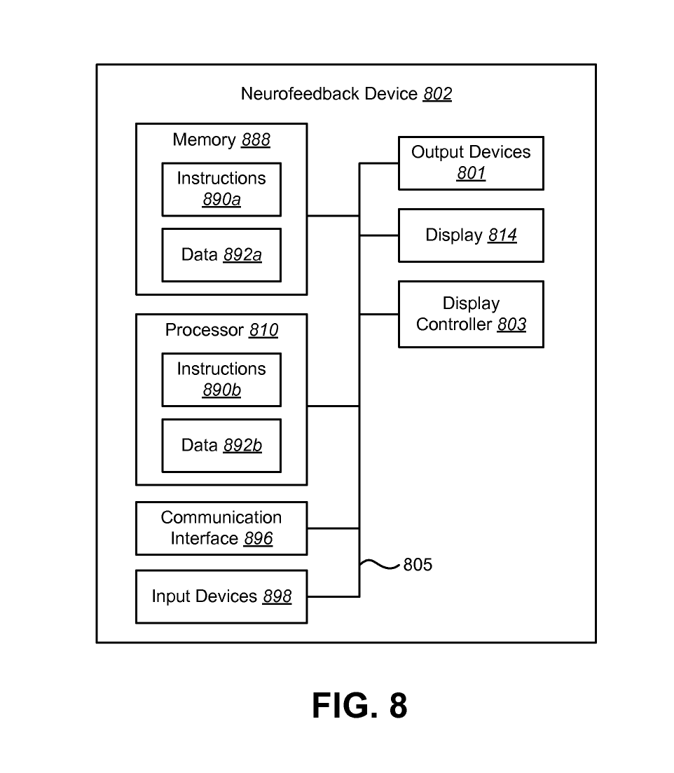

FIG. 8 illustrates various components that may be utilized in a neurofeedback device 802. The neurofeedback device 802 may be utilized as the neurofeedback device 102, 402, 502, 702 illustrated previously. The neurofeedback device 802 includes a processor 810 that controls operation of the neurofeedback device 802. The processor 810 may also be referred to as a CPU. Memory 888, which may include both read-only memory (ROM), random access memory (RAM) or any type of device that may store information, provides instructions 890a and data 892a to the processor 810. A portion of the memory 888 may also include non-volatile random access memory (NVRAM). Instructions 890b and data 892b may also reside in the processor 810. Instructions 890b and/or data 892b loaded into the processor 810 may also include instructions 890a and/or data 892a from memory 888 that were loaded for execution or processing by the processor 810. The instructions 890b may be executed by the processor 810 to implement the systems and methods disclosed herein.

The neurofeedback device 802 may include one or more communication interfaces 896 for communicating with other electronic devices. The communication interfaces 896 may be based on wired communication technology, wireless communication technology, or both. Examples of communication interfaces 896 include a serial port, a parallel port, a Universal Serial Bus (USB), an Ethernet adapter, an IEEE 1394 bus interface, a small computer system interface (SCSI) bus interface, an infrared (IR) communication port, a Bluetooth wireless communication adapter, and so forth.

The neurofeedback device 802 may include one or more output devices 801 and one or more input devices 898. Examples of output devices 801 include a speaker, printer, etc. One type of output device that may be included in a neurofeedback device 802 is a display device 814. Display devices 814 used with configurations disclosed herein may utilize any suitable image projection technology, such as a cathode ray tube (CRT), liquid crystal display (LCD), light-emitting diode (LED), gas plasma, electroluminescence or the like. A display controller 803 may be provided for converting data stored in the memory 888 into text, graphics and/or moving images (as appropriate) shown on the display 814. Examples of input devices 898 include a keyboard, mouse, microphone, remote control device, button, joystick, trackball, touchpad, touchscreen, lightpen, etc.

The various components of the neurofeedback device 802 are coupled together by a bus system 805, which may include a power bus, a control signal bus and a status signal bus, in addition to a data bus. However, for the sake of clarity, the various buses are illustrated in FIG. 8 as the bus system 805. The neurofeedback device 802 illustrated in FIG. 8 is a functional block diagram rather than a listing of specific components.

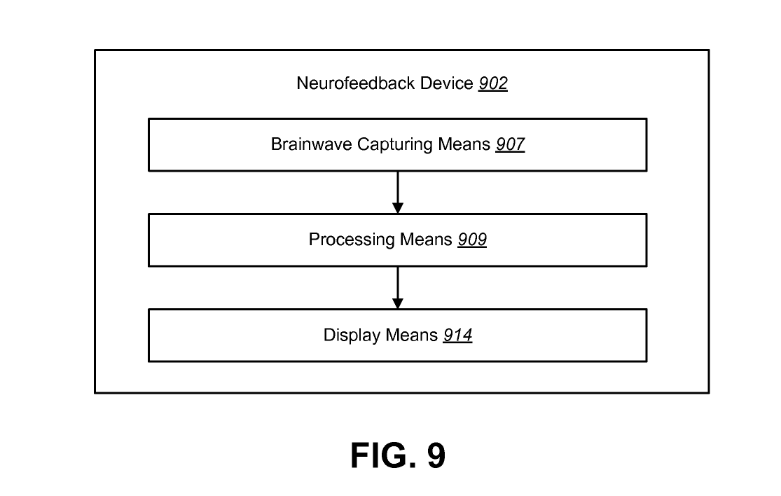

FIG. 9 is a block diagram illustrating another configuration of a neurofeedback device 902 in which systems and methods for portable neurofeedback may be implemented. The neurofeedback device 902 includes brainwave capturing means 907, processing means 909 and display means 914. The brainwave capturing means 907, processing means 909 and display means 914 may be configured to perform one or more of the functions described in connection with FIG. 1, FIG. 2 and FIG. 8 above. FIG. 8 above illustrates one example of a concrete apparatus structure of FIG. 9. Other various structures may be implemented to realize one or more of the functions of FIG. 1, FIG. 2 and FIG. 8.

The term “computer-readable medium” refers to any available medium that can be accessed by a computer or a processor. The term “computer-readable medium,” as used herein, may denote a computer- and/or processor-readable medium that is non-transitory and tangible. By way of example, and not limitation, a computer-readable or processor-readable medium may comprise RAM, ROM, Electrically Erasable Programmable Read-Only Memory (EEPROM), Compact Disc Read-Only Memory (CD-ROM) or other optical disk storage, magnetic disk storage or other magnetic storage devices, or any other medium that can be used to carry or store desired program code in the form of instructions or data structures and that can be accessed by a computer or processor. Disk and disc, as used herein, includes compact disc (CD), laser disc, optical disc, digital versatile disc (DVD), floppy disk and Blu-ray® disc where disks usually reproduce data magnetically, while discs reproduce data optically with lasers.

It should be noted that one or more of the methods described herein may be implemented in and/or performed using hardware. For example, one or more of the methods described herein may be implemented in and/or realized using a chipset, an application-specific integrated circuit (ASIC), a large-scale integrated circuit (LSI) or integrated circuit, etc.

Each of the methods disclosed herein comprises one or more steps or actions for achieving the described method. The method steps and/or actions may be interchanged with one another and/or combined into a single step without departing from the scope of the claims. In other words, unless a specific order of steps or actions is required for proper operation of the method that is being described, the order and/or use of specific steps and/or actions may be modified without departing from the scope of the claims.

It is to be understood that the claims are not limited to the precise configuration and components illustrated above. Various modifications, changes and variations may be made in the arrangement, operation and details of the systems, methods, and apparatus described herein without departing from the scope of the claims.

What is claimed is:

1. A portable neurofeedback device, comprising:
a wearable fixture;
one or more brainwave sensors attached to the wearable fixture;
a processing unit attached to the wearable fixture, wherein the processing unit receives brainwave signals captured by the one or more brainwave sensors, and computes one or more user-feedback values that represent current brain activity; and
a display module attached to the wearable fixture that displays a visual representation of the one or more user-feedback values.

2. The neurofeedback device of claim 1, wherein the visual representation of the one or more user-feedback values is displayed in real time.

3. The neurofeedback device of claim 1, wherein the display module is only visible to a user wearing the neurofeedback device.

4. The neurofeedback device of claim 3, wherein the display module comprises at least one of a light emitting diode (LED), an LED array, a multi-color LED array and a liquid crystal display (LCD).

5. The neurofeedback device of claim 1, further comprising multiple algorithms used by the processing unit to compute the one or more user-feedback values, wherein each algorithm produces a different user-feedback value, and wherein the multiple algorithms correspond to different types of neurofeedback training.

6. The neurofeedback device of claim 5, further comprising a selection means attached to the neurofeedback device for selecting one or more of the multiple algorithms.

7. The neurofeedback device of claim 1, further comprising one or more communication interfaces to communicate with at least one external device.

8. The neurofeedback device of claim 7, wherein the at least one external device displays a visual representation of the one or more user-feedback values and saves log data.

9. The neurofeedback device of claim 7, wherein the at least one external device further comprises a user-interface for configuring the neurofeedback device.

10. The neurofeedback device of claim 1, wherein the wearable fixture is a cap, comprising:
a crown portion that includes the one or more brainwave sensors; and
a brim portion that projects from the crown portion, wherein the display module is attached to a bottom side of the brim portion and is visible by a user wearing the neurofeedback device.

11. The neurofeedback device of claim 1, wherein the wearable fixture comprises:
a headband portion that includes the one or more brainwave sensors; and
an eyeglass portion, wherein the display module is attached to the eyeglass portion and is visible by a user wearing the neurofeedback device.

12. The neurofeedback device of claim 1, wherein the user-feedback value corresponds to a power ratio of two or more frequency bands.

13. A method for performing neurofeedback by a portable neurofeedback device, comprising:
capturing, by one or more brainwave sensors attached to a wearable fixture, brainwave signals;
computing, by a processing unit attached to the wearable fixture, one or more user-feedback values that represent current brain activity; and
displaying, by a display module attached to the wearable fixture, a visual representation of the one or more user-feedback values.

14. The method of claim 13, wherein the visual representation of the one or more user-feedback values is displayed in real time.

15. The method of claim 13, wherein the display module is only visible to a user wearing the neurofeedback device.

16. The method of claim 15, wherein the display module comprises at least one of a light emitting diode (LED), an LED array, a multi-color LED array, and a liquid crystal display (LCD).

17. The method of claim 13, further comprising using multiple algorithms to compute the one or more user-feedback values, wherein each algorithm produces a different user-feedback value, and wherein the multiple algorithms correspond to different types of neurofeedback training.

18. The method of claim 13, further comprising communicating with at least one external device.

19. The method of claim 18, further comprising:
displaying, by the at least one external device, a visual representation of the one or more user-feedback values; and
saving, by the at least one external device, log data.

20. The method of claim 13, wherein the user-feedback value corresponds to a power ratio of two or more frequency bands.
